(19) United States
(12) Patent Application Publication
(10) Pub. No.: US 2026/0232907 A1
(43) Pub. Date: Aug. 13, 2026

(54) DRUG INJECTION DEVICE

(71) Applicant: EUNSUNG GLOBAL CORP, Wonju-si (KR)

(72) Inventor: Ki Se LEE, Seoul (KR)

(21) Appl. No.: 19/322,976

(22) Filed: Sep. 9, 2025

(30) Foreign Application Priority Data
Feb. 13, 2025 (KR) 10-2025-0018865
Aug. 21, 2025 (KR) 10-2025-0116185

(51) Int. Cl. A61M 5/20; A61M 5/31; A61M 5/32

(52) U.S. Cl. CPC A61M 5/2053; A61M 5/3145; A61M 5/3202

(57) ABSTRACT

Proposed is a drug injection device including a gun-type housing configured to include a grip part, an operation part, and a mounting part, and to have an interior space that is spatially connectable with a suction device, a syringe placed on the mounting part, a drug injector mounted on an edge of the mounting part, configured to supply a drug by communicating with the syringe, provided with a flow path spatially connected to the interior space of the gun-type housing, and configured to quantitatively inject a drug by improving adhesion to skin due to a vacuum generation part provided between the skin and a front surface thereof by an operation of the suction device, a suction connection part spatially connectable with the suction device, and an air filtering unit having at least a portion thereof spatially connected to the flow path to filter suction air in the interior space.

### CROSS REFERENCE TO RELATED APPLICATION

The present application claims priority to Korean Patent Application No. 10-2025-0018865, filed Feb. 13, 2025, and Korean Patent Application No. 10-2025-0116185, filed Aug. 21, 2025, the entire contents of which is incorporated herein for all purposes by this reference.

### BACKGROUND

### Technical Field

The present disclosure relates to a drug injection device for injecting medication under the skin for skin treatment.

### Description of the Related Art

The function of the human skin tissue can be impaired by aging and ultraviolet (UV) radiation, leading to various changes. Representative changes include wrinkle formation due to reduction and deformation of collagen fibers in the dermis, a decrease in skin elasticity due to degeneration of elastic fibers composed of elastin, and melanin pigmentation due to activation of tyrosinase in the epidermal layer.

To prevent skin aging and maintain and improve elasticity, a non-chemical method called electroporation has been used to introduce effective substances into cells to induce beneficial biological changes.

Meanwhile, drug injection methods based on the principle of electroporation are mainly used for skincare and plastic surgery, and for treatment of specific parts of the body.

Electroporation, used in skin treatment, is a technique that utilizes electrical pulses to disrupt the phospholipid bilayer, creating temporary pores in the cell membrane of the skin's outer layer, allowing substances like drugs or other molecules with a molecular weight less than 4000 Daltons to enter the cell.

The above information may be provided as background art for the purpose of assisting in understanding the present disclosure. No claim or determination is made as to whether any of the above is applicable as related art in connection with the present disclosure.

### Document of Related Art

(Patent Document 1) Korean Patent No. 10-0420731

### SUMMARY

When injecting the required drug into skin cells using a drug injection device, it can be challenging to inject a precise amount of a drug.

If the drug being injected into the skin cells is inexpensive, this may not matter, but if the drug is expensive, precise dosing of the drug can be very important.

In practice, there are many cases where drugs are excessively injected into skin cells, and expensive drugs are unnecessarily consumed in excess of the recommended amount, which can be uneconomical from the consumer's perspective.

Technical problems to be solved by the present disclosure need not be limited to the ones mentioned above, and other technical problems not mentioned can be clearly understood by those with ordinary knowledge in the technical field to which the present disclosure belongs from the description below.

In order to solve the above problems, according to an aspect of the present disclosure, there is provided a drug injection device that enables of quantitative injection of drugs.

In addition, according to another aspect of the present disclosure, there is provided a drug injection device that enables quantitative injection of drugs by using the front space between the skin and the needle cover as a suction space.

In addition, according to another aspect of the present disclosure, there is provided a drug injection device that is economical from the consumer's point of view since the device minimizes unnecessary drug consumption by quantitatively injecting expensive drugs.

According to various embodiments, a drug injection device may include: a gun-type housing configured to include a grip part, an operation part adjacent to the grip part, and a mounting part located above the grip part, and to have an interior space that is spatially connectable with a suction device; a syringe placed on the mounting part; a drug injector mounted on an edge of the mounting part, configured to supply a drug by communicating with the syringe, provided with a flow path spatially connected to the interior space of the gun-type housing, and configured to quantitatively inject a drug by improving adhesion to skin due to a vacuum generation part provided between the skin and a front surface thereof by an operation of the suction device; a suction connection part disposed at an edge of the gun-type housing and spatially connectable with the suction device; and an air filtering unit disposed in the gun-type housing and configured to have at least a portion thereof spatially connected to the flow path to filter suction air in the interior space.

According to various embodiments, a drug injection device may include: a gun-type housing configured to include a grip part, an operation part adjacent to the grip part, and a mounting part located above the grip part, and to have an interior space that is spatially connectable with a suction device; a syringe placed on the mounting part; a drug injector mounted on an edge of the mounting part, configured to supply a drug by communicating with the syringe, provided with a flow path spatially connected to the interior space of the gun-type housing, and configured to quantitatively inject a drug by improving adhesion to skin due to a vacuum generation part provided between the skin and a front surface thereof by an operation of the suction device; a suction connection part disposed at an edge of the gun-type housing and connectable with the suction device; an air filtering unit disposed in the gun-type housing and configured to have at least one end thereof disposed in the interior space, spatially connected to the flow path, and configured to filter suction air in the interior space; and at least one air tube physically and spatially connecting between the suction connection part, the air filtering unit, and the drug injector.

According to an embodiment of the present disclosure, by using the front space between the skin and a needle cover as a suction space, precise quantitative injection of expensive drugs is possible, thereby minimizing the consumption of expensive drugs that are unnecessarily injected.

Since the effects of the present disclosure described above are naturally exerted by the composition of the described contents regardless of whether the inventor is aware of them, the above-described effects are only some of the effects according to the described contents and should not be recognized as describing all the effects recognized or actually existing by the inventor.

In addition, the effects of the present disclosure should be additionally understood by the entire description of the specification, and even if it is not explicitly described in a sentence, if it is an effect that can be recognized as such by a person with common knowledge in the technical field to which the described content belongs through this specification, it should be considered an effect described in this specification.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a perspective view showing a drug injection device from one side according to an embodiment;

FIG. 2 is a perspective view showing a drug injection device from the other side according to an embodiment;

FIG. 3 is a side view showing a drug injection device according to an embodiment;

FIG. 4 is an exemplary drawing showing the state of mounting an air filtering unit of a drug injection device according to an embodiment;

FIG. 5 is a perspective view showing the front of a drug injector according to an embodiment;

FIG. 6 is a perspective view showing the rear of a drug injector according to an embodiment;

FIG. 7 is an exploded perspective view showing a drug injector according to an embodiment;

FIG. 8 is a perspective view showing the front of a needle frame according to an embodiment;

FIG. 9 is a perspective view showing the rear of a needle frame according to an embodiment;

FIG. 10 is a perspective view showing a needle housing before assembly according to an embodiment;

FIG. 11 is a cross-sectional view showing a combined state of a needle frame and a needle housing after assembly according to an embodiment;

FIG. 12 is a cross-sectional view of an assembled drug injector according to an embodiment;

FIG. 13A is a perspective view showing a duct structure according to an embodiment;

FIG. 13B is a cross-sectional view showing a duct structure according to an embodiment;

FIG. 14 is a perspective view showing an air filtering unit according to an embodiment;

FIG. 15 is a perspective view showing a suction device according to an embodiment;

FIG. 16 is a perspective view showing a suction device having a drug injection device mounted thereon according to an embodiment;

FIG. 17 is a side view showing a drug injection device according to an embodiment;

FIG. 18 is a perspective view showing the front of a drug injector according to an embodiment;

FIG. 19 is a perspective view showing the rear of a drug injector according to an embodiment;

FIG. 20 is an exploded perspective view showing a drug injector with a support frame separated from a needle cover according to an embodiment;

FIG. 21 is an exploded perspective view showing a drug injector according to an embodiment;

FIG. 22 is a perspective view showing the front of a needle frame according to an embodiment;

FIG. 23 is a perspective view showing the rear of a needle frame according to an embodiment;

FIG. 24 is a perspective view showing a needle housing before assembly according to an embodiment;

FIG. 25 is a cross-sectional view showing a combined state of a needle frame and a needle housing after assembly according to an embodiment;

FIGS. 26 and 27 are cross-sectional views each showing an assembled drug injector according to an embodiment;

FIG. 28 is an enlarged view of a portion of a cutaway view showing the state in which a contact elastic member is arranged on the front surface of a needle cover according to an embodiment; and

FIG. 29 is a view showing the suction state of a contact elastic member illustrated in FIG. 28.

Regarding the description of the drawings, the same or similar reference numerals may be used for identical or similar components.

### DETAILED DESCRIPTION

Hereinafter, embodiments will be described in detail with reference to the attached drawings so that those skilled in the art can easily implement the present disclosure. However, the disclosed embodiments may be implemented in various different forms and are not limited to the embodiments described herein.

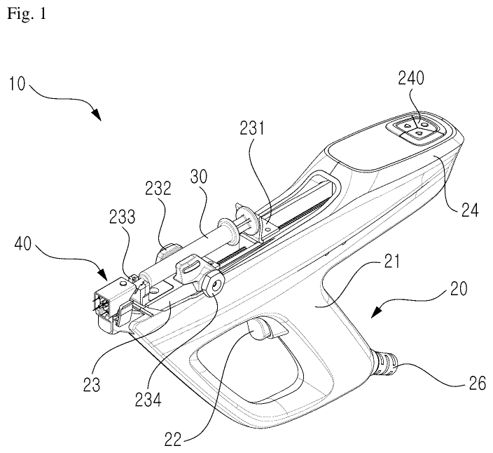

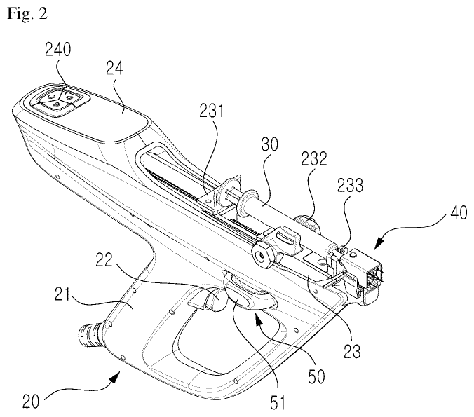

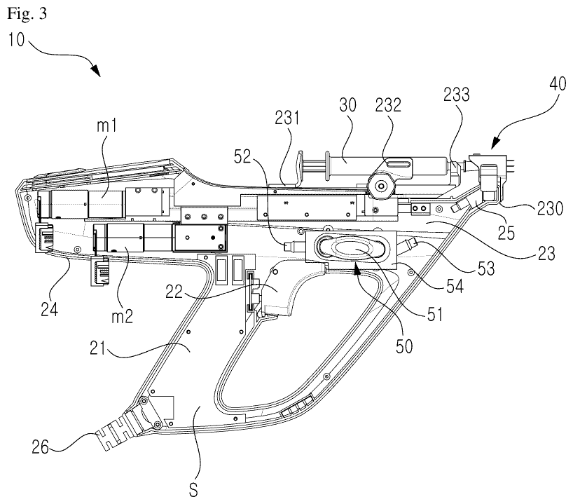

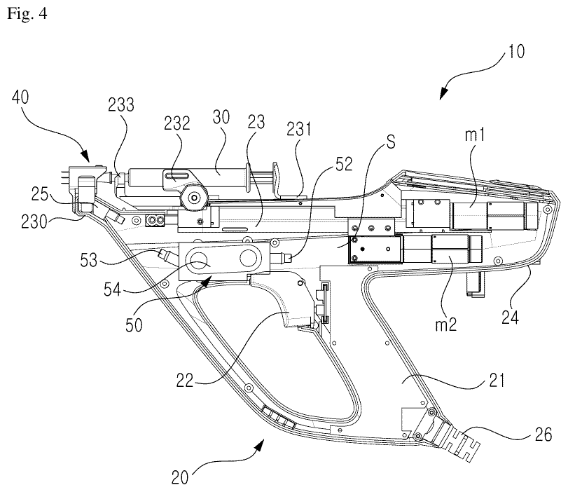

FIG. 1 is a perspective view showing a drug injection device from one side according to an embodiment. FIG. 2 is a perspective view showing a drug injection device from the other side according to an embodiment. FIG. 3 is a side view showing a drug injection device according to an embodiment. FIG. 4 is an exemplary drawing showing the state of mounting an air filtering unit of a drug injection device according to an embodiment.

Referring to FIGS. 1 to 4, according to an embodiment, a drug injection device 10 is a device that can quantitatively inject drugs into skin cells in environments such as hospitals, homes, and emergency situations, and may be a gun-type device that can quantitatively inject expensive drugs in particular. According to an embodiment, the drug injection device 10 may be an injection device that can inject a fixed amount of drug into the human skin, which is a free surface, using a suction device. For example, the drug injection may include intravenous injection, subcutaneous injection, or intramuscular injection.

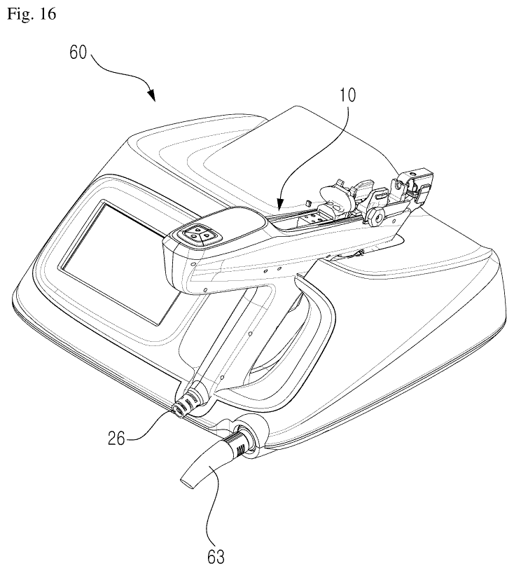

According to an embodiment, the drug injection device 10 may include a gun-type housing 20, a syringe 30, a slider 231, a drug injector 40 and an air filtering unit 50. According to an embodiment, the gun-type housing 20 may be physically connected to a suction device (e.g., the suction device 60 illustrated in FIG. 16), and has an internal space s that can be spatially connected to the suction device. The internal space s may be converted into a suction space by the suction device.

According to an embodiment, the gun-type housing 20 may include a grip part 21, an operation part 22, a mounting part 23 in which the syringe 30 and the drug injector 40 are mounted, and a rear part 24 in which a keypad 240 is placed. According to an embodiment, the gun-type housing 20 may have the grip part 21 and the operation part 22 provided beneath the mounting part 23, and have the mounting part 23 provided on the upper portion thereof and the rear part 24 at the rear of the mounting part 23.

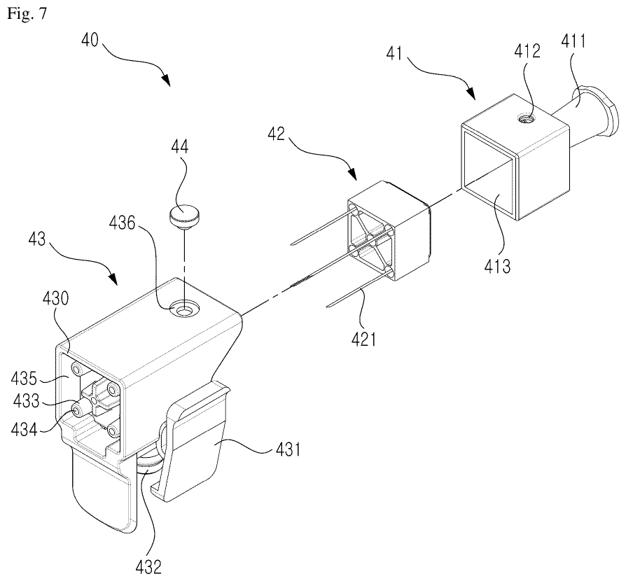

According to an embodiment, the gun-type housing 20 may have the slider 231, a holder 232, and a support 233 arranged along the upper portion of the mounting part 23. According to an embodiment, the syringe 30 may be held by the holder 232 and maintained in a state of being pressed toward the drug injector 40 by the slider 231 supported by a spring. According to an embodiment, the end of the syringe 30 spatially connected to an injection port (e.g., the injection port 411 shown in FIG. 7) of the drug injector 40 while being supported on the support 233, so that a drug stored in the syringe 30 may be supplied to the drug injector 40.

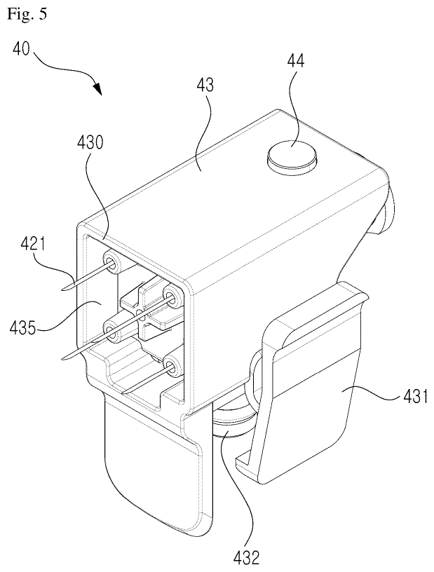

According to an embodiment, the drug injector 40 may be mounted on an edge 230 of the mounting part 23. According to an embodiment, the drug injector 40 may be fixed to the edge 230 of the mounting part 23 by having a pair of hook parts (e.g., the hook part 431 illustrated in FIG. 5). Reference numeral 234 may be a member that adjusts the distance between the pair of holders 232 to adjust the holding strength of the mounted syringe.

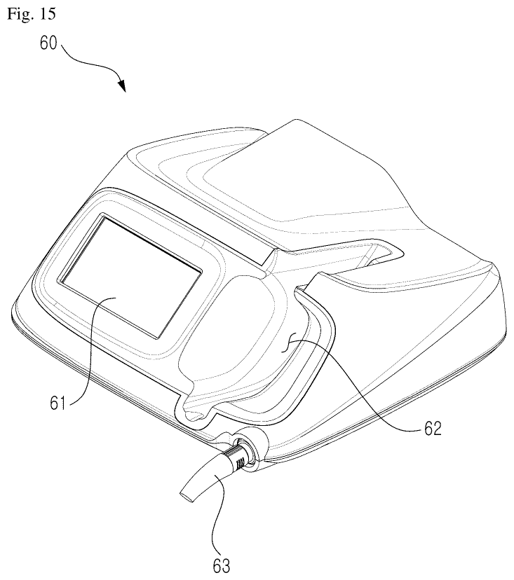

According to an embodiment, the edge of the gun-type housing 20 may include a suction connection part 26 for being physically and spatially connected to a suction device (e.g., the suction device 60 illustrated in FIG. 15).

According to an embodiment, the syringe 30 is a device that quantitatively discharges a drug into the drug injector 40, and may be used in a pumping manner to push out a drug by a pressure of a tube.

Referring to FIGS. 2 to 4, according to an embodiment, the gun-type housing 20 may include the air filtering unit 50. According to an embodiment, the air filtering unit 50 may be arranged in the gun-type housing 20 and may filter the suction air in the internal space s of the gun-type housing 20. According to an embodiment, the air filtering unit 50 may be arranged between the mounting part 23 and the operation part 22 of the gun-type housing 20.

According to an embodiment, the air filtering unit 50 may may be partially exposed to the outside of the gun-type housing 20 and the remaining part may be located inside the gun-type housing 20. According to an embodiment, the air filtering unit 50 may include a first part 51 positioned to be exposed to the outside of the gun-type housing 20 and a second part 52 and 53 positioned inside the gun-type housing 20. According to an embodiment, the air filtering unit 50 may be located between the suction connection part 26 and the drug injector 40 and may filter suction air passing through a suction flow path between the suction connection part 26 and the drug injector 40.

According to an embodiment, the first part 51 may have a convex shape, and the second part 52 and 53 may have a tube shape. According to an embodiment, the second part 52 and 53 may include a first tubular part 52 arranged toward the suction connection part 26, and a second tubular part 53 spaced apart from the first tubular part 52 but directed toward the drug injector 40.

For example, an air flow path may be formed between the suction connection part 26 and the second tubular part 53, an air flow path may also be formed between the tubular part 53 and the first tubular part 52, and an air flow path may also be formed between the first tubular part 52 and the drug injector 40. In the case that the gun-type housing 20 is connected to the suction device (e.g., the suction device 60 shown in FIG. 16) so that the internal space s is suctioned, the air flow path may be a suction flow path.

According to an embodiment, the cross-sectional area of the first part 51 may be formed larger than the cross-sectional area of the second part 52 or 53.

According to an embodiment, the gun-type housing 20 may be equipped with a plurality of drive motors m1 and m2 to inject a drug at a set speed and amount. In an embodiment, the drive motors may include a first drive motor m1 associated with drug injection and a second drive motor m2 associated with needle operation. In an embodiment, the first and second drive motors m1 and m2 may be positioned close to each other, spaced apart from each other, and disposed at the rear portion of the gun-type housing.

According to an embodiment, the first drive motor m1 is a driving unit that has the function of delivering a fixed amount of drug at a constant rate for a fixed period of time, and the drug injection speed may be controlled by precisely controlling the rotation speed of the motor.

According to an embodiment, the second drive motor m2 needs to precisely control the position of the piston or plunger of the syringe 30, thereby enabling quantitative drug injection control through precise rotation speed control.

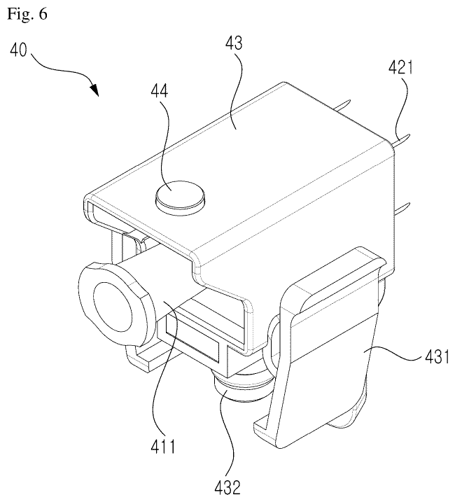

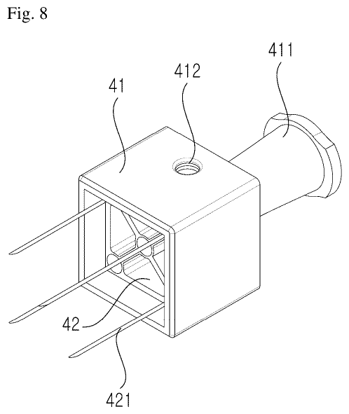

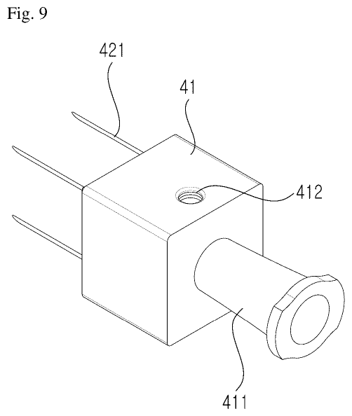

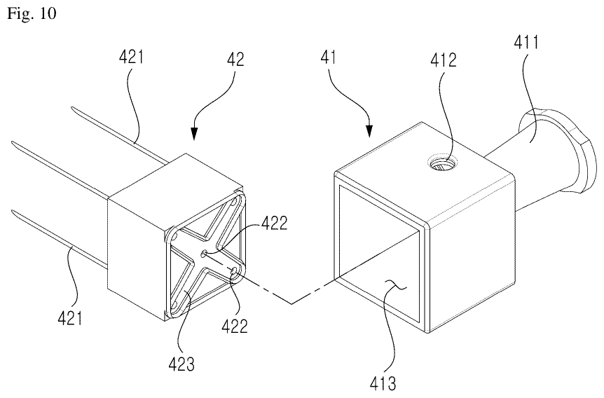

FIG. 5 is a perspective view showing the front of a drug injector according to an embodiment. FIG. 6 is a perspective view showing the rear of a drug injector according to an embodiment. FIG. 7 is an exploded perspective view showing a drug injector according to an embodiment. FIG. 8 is a perspective view showing the front of a needle frame according to an embodiment. FIG. 9 is a perspective view showing the rear of a needle frame according to an embodiment. FIG. 10 is a perspective view showing a needle housing before assembly according to an embodiment.

Referring to FIGS. 5 to 10, the structure of the drug injector 40 according to an embodiment will be described.

Referring to FIGS. 5 to 10, the drug injector 40 may include a needle frame 42, a needle housing 41, a needle cover 43, and a fastening structure 44, 412. According to an embodiment, the needle frame 42 is a member that supports a plurality of needles 421, and may have one side that is open and the other side that is closed. According to an embodiment, the needle frame 42 may have the plurality of needles 421 arranged in an open space on one side, and a drug passage 423 formed on the other side through which the supplied drug flows.

For example, the arrangement of the needles 421 need not be limited to a 5-pin type, and may be changed to a 9-pin type. According to an embodiment, the drug passage 423 is formed in a grooved cross shape and is connected to each needle hole 422, so that the supplied drug can flow to each needle 421. According to an embodiment, the needle frame 42 is provided with five needle holes 422, and each needle 421 may be fixed to each needle hole 422.

According to an embodiment, the needle housing 41 is a housing member for accommodating the needle frame 42 within an interior 413 thereof, and may have one side open to accommodate the needle frame 42 while the other side formed with an injection part 411 that is physically connected to a syringe (e.g., the syringe 30 illustrated in FIG. 1). According to an embodiment, the needle housing 41 has a fastening hole 412 formed on one side, and when the needle housing 41 inserted into the needle cover 43, a fastening member 44 is fastened to the fastening hole 412, so that the needle housing 41 may be restrained by the needle cover 43.

According to an embodiment, the needle cover 43 has a plurality of needle guides 433 arranged in a protruding shape on one side to support the each individual needle 421, and the other side is open so that the needle housing 41 into which the needle frame 42 is inserted may be accommodated. Each needle guide 433 may have a through hole 434 through which the needle 421 passes.

According to an embodiment, the needle cover 43 is formed with the elastic hook parts 431 facing each other, so that the needle cover 43 may be mounted on the edge of the mounting part 23. According to an embodiment, the needle cover 43 has a fastening hole formed on one side, and the fastening member is fastened to the fastening hole, so that the needle cover 43 and the needle housing 41 may be assembled as one piece.

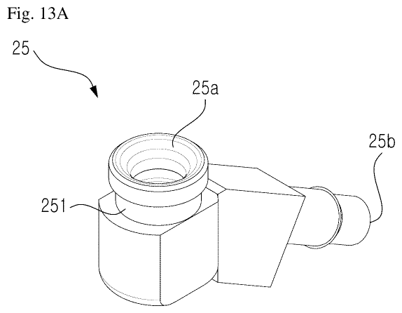

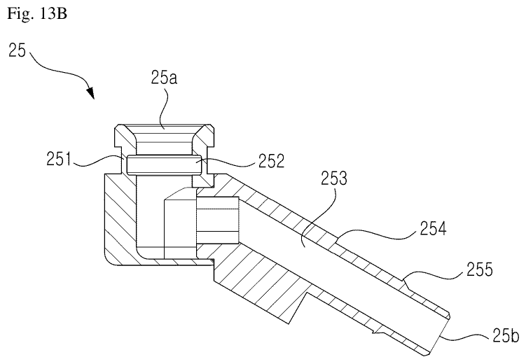

According to an embodiment, the needle cover 43 may include a downwardly extending connection part 432. According to an embodiment, the connection part 432 may be physically connected and spatially connected to a duct structure described below (e.g., the duct structure 25 illustrated in FIG. 13).

According to an embodiment, when the needle cover 43 comes into contact with the skin in a state in which the needle housing 41 with the needle frame 42 combined is inserted into the needle cover 43, a sealed front space 435 is provided between the front surface 430 of the needle cover 43 and the skin, and the sealed front space 435 may converted into a suction space by a suction device (e.g., the suction device 60 illustrated in FIG. 16).

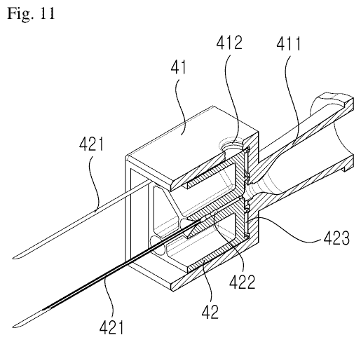

FIG. 11 is a cross-sectional view showing a combined state of a needle frame and a needle housing after assembly according to an embodiment.

Referring to FIG. 11, according to an embodiment, the needle housing 41 may be supplied with a drug through an injection part 411 connected to the syringe 30. According to an embodiment, the drug supplied through the injection part 411 flows into the five needle holes 422 through the drug passage 423 formed on the other surface of the needle frame 42, and the drug flowing into each needle hole 422 may be supplied to each needle 421.

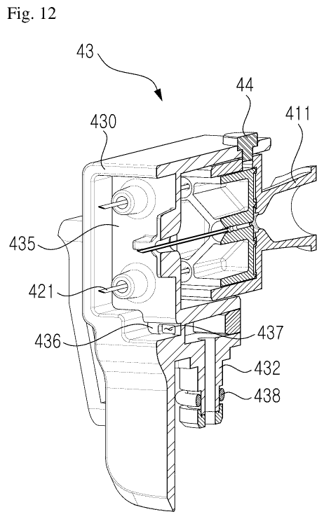

FIG. 12 is a cross-sectional view of an assembled drug injector according to an embodiment.

Referring to FIG. 12, when the drug injector 40 comes into contact with the skin, that is, when the front surface 430 of the needle cover 43 comes into contact with the skin, the front space 435 is sealed by the skin, so that a vacuum generation part may be formed in the front space 435. According to an embodiment, the front space 435 is a space where suction is possible, and the adhesion to the skin is improved by the vacuum generation part to enable quantitative injection of the drug, and a path for suction may be formed using a suction device. According to an embodiment, a flow path hole 437 is formed in a wall 436 between the front space 435, which is formed by contact with the skin and the front surface 430 of the needle cover 43, and the connection part 432, so that a path through which suction air flows may be formed. According to an embodiment, the path is a pathway through which suction air flows from the front space 435 to the connection part 432, and may be used as a flow path for suction. Reference numeral 438 may be a waterproof member.

FIG. 13A is a perspective view showing a duct structure according to an embodiment. FIG. 13B is a cross-sectional view showing a duct structure according to an embodiment.

Referring to FIGS. 3, 4, 13A, and 13B, according to an embodiment, the drug injector 40 may include the duct structure 25 physically and spatially connected to the flow path. According to an embodiment, the duct structure 25 is physically and spatially connected to the connection part 432 of the needle cover 43, so that the duct structure 25 may be a part of the flow path through which air or suction air of the internal space s flows. According to an embodiment, an inlet 25a of the duct structure 25 may be spatially connected to the flow path of the drug injector, and an outlet 25b may be spatially connected to the internal space s of the gun-type housing 20.

According to an embodiment, a waterproof structure may be arranged between the connection part 432 and the duct structure 25. According to an embodiment, the waterproof structure may include: a waterproof member (e.g., the waterproof member 438 illustrated in FIG. 12) arranged in a pressurized state on the outer surface of the connecting part 432; and a seating recess 252 formed on the inner surface of an inlet part 251 of the duct structure 25 and in which the waterproof member 438 is seated. For example, the waterproof member 438 and the seating recess 252 may be ring-shaped.

According to an embodiment, the duct structure 25 may include a flow path part 253 arranged to face the air filtering unit 50. For example, the flow path part 253 has a structure that extends in a straight line, but is not limited to a straight line, and a curved shape may also be changed.

According to an embodiment, the duct structure 25 may include a step 254 to prevent excessive insertion when mounted on an edge (e.g., the edge 230 illustrated in FIG. 1) of the mounting part 23. According to an embodiment, the duct structure 25 may include a catch protrusion 255 for mounting on the edge 230 of the mounting part 23. For example, the catch protrusion 255 may be ring-shaped.

According to an embodiment, although the duct structure 25 is exemplified as being manufactured separately and connected to the connection part of the drug injector 40, it is not necessary to be limited to this structure, and the duct structure 25 may be formed by injection molding integrally with the connection part of the drug injector 40.

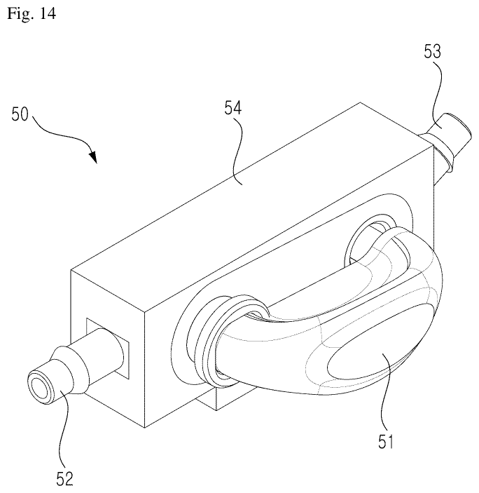

FIG. 14 is a perspective view showing an air filtering unit according to an embodiment.

Referring to FIG. 14, the air filtering unit 50 according to an embodiment may be supported at least in part by a box-shaped housing 54 on the gun-type housing 20. According to an embodiment, the air filtering unit 50 may have a filter part for an air filter arranged in the first part 51. According to an embodiment, the air filtering unit 50 may be a part of a suction flow path and may filter the suction air passing therethrough.

According to an embodiment, the mounting position of the air filtering unit 50 may be anywhere where a suction flow path is formed. In this embodiment, although the air filtering unit 50 is exemplified as being placed between the mounting part 23 and the operation part 22 of the gun-type housing, it is not necessary to be limited thereto, and the air filtering unit 50 may be placed at any point where a suction flow path is formed.

In addition, although in this embodiment, the air filtering unit 50 is exemplified as being partly positioned outside the gun-type housing 20 and partly positioned in the internal space s of the gun-type housing, it is not necessary to be limited thereto, and a structure in which the air filtering unit 50 may be placed in the internal space s of the gun-type housing may also be possible.

According to an embodiment, the air filtering unit 50 may be responsible for removing impurities from the suction air passing through. According to an embodiment, the air filtering unit 50 is configured to be detachable from the gun-type housing 20, so that the air filtering unit 50 can be replaced.

FIG. 15 is a perspective view showing a suction device according to an embodiment. FIG. 16 is a perspective view showing a suction device having a drug injection device mounted thereon according to an embodiment.

Referring to FIGS. 15 and 16, according to an embodiment, the drug injection device 10 may be physically and spatially connected to the suction device 60 for suctioning of the sealed front space 435 between the skin and the front surface of the needle housing 41. According to an embodiment, the suction connection part 26 is arranged at an edge of the gun-type housing 20, and the suction connection part 26 may be spatially connected to the suction device 60. According to an embodiment, the suction device 60 may have a display part 61 and a mounting slot 62 arranged in the front thereof. According to an embodiment, the suction device 60 may include a suction connection part 63 for spatially connecting with the drug injection device 10. According to an embodiment, the suction connection part 63 may be spatially connected with the suction connection part 26 of the gun-type housing 20. According to an embodiment, the mounting slot 62 may be formed in a shape corresponding to at least a portion of the gun-type housing 20. According to an embodiment, when the suction device 60 operates, the internal space of the gun-type housing (e.g., the internal space s illustrated in FIG. 3) is suctioned, and the flow path of a drug injector (e.g., the drug injector 40 illustrated in FIG. 1) spatially connected to the internal space s is suctioned, so that the front space 435 in contact with the skin may maintain a suction state, and subsequently, a fixed amount of drug may be injected using a needle.

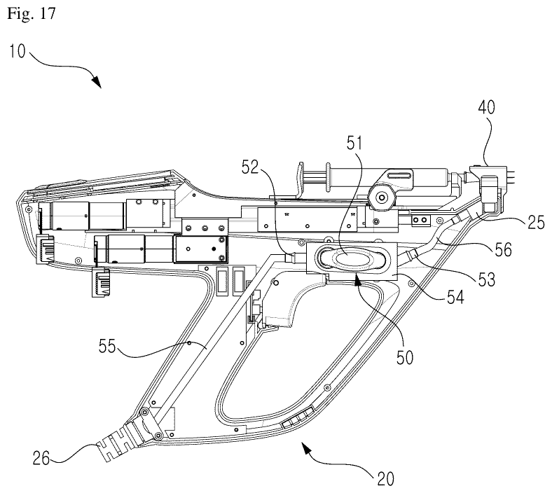

FIG. 17 is a side view showing a drug injection device according to an embodiment.

Referring to FIG. 17, according to an embodiment, the drug injection device 10 may form a new suction flow path by using at least one air tube 55 or 56. According to an embodiment, the air filtering unit 50 may be connected to at least one air tube 55 or 56. According to an embodiment, the first tubular part 52 of the air filtering unit 50 may be physically and spatially connected to the suction connection part 26 using a first air tube 55. According to an embodiment, the second tubular part 53 of the air filtering unit 50 may be physically and spatially connected to the duct structure 25 using a second air tube 56.

According to an embodiment, the suction flow path consisting of the suction connection part 63 and 26, the first air tube 55, the first tubular part 52, the first part 51, the second tubular part 53, the duct structure 25, the flow path, and a front space (e.g., the front space 435 illustrated in FIG. 5) of the drug injector 40 may be formed by the suction device 60. According to an embodiment, the first part 51 of the air filtering unit 50 may be replaced with an air tube, and a filter may be placed inside the air tube.

According to an embodiment, the interior space of the suction device 60 may be provided with a printed circuit board (not shown), a switched mode power supply (SMPS), a vacuum pump, three solenoid valves for pressure control, an air filter, etc.

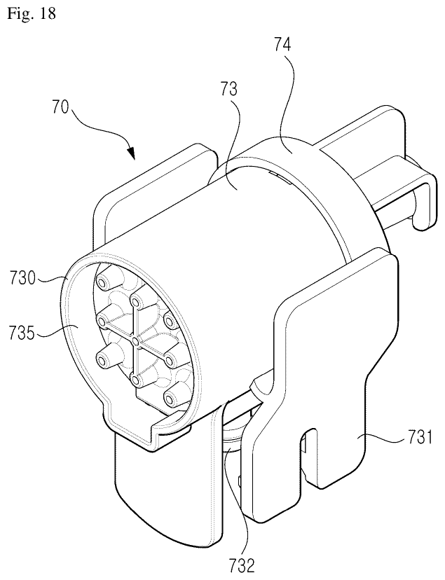

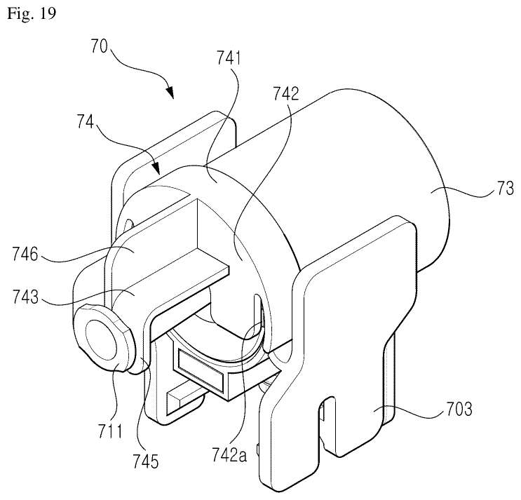

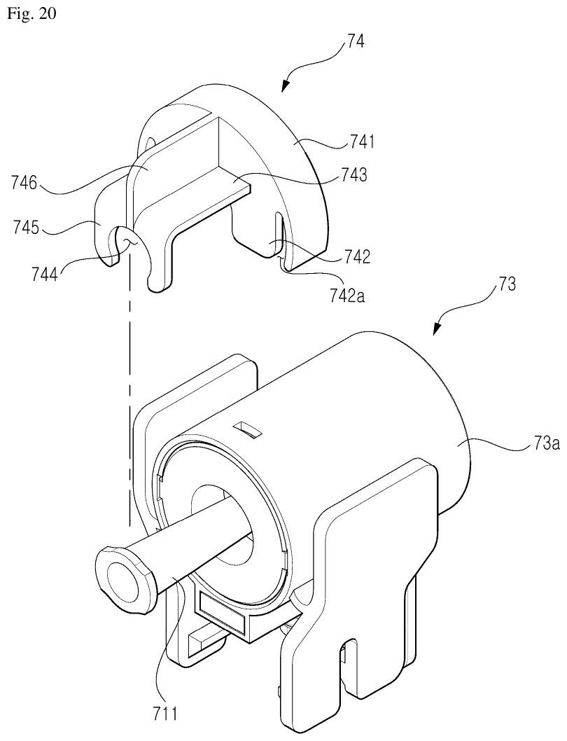

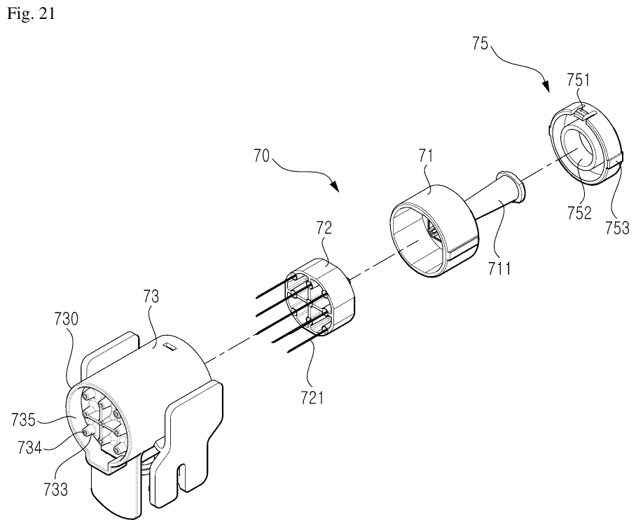

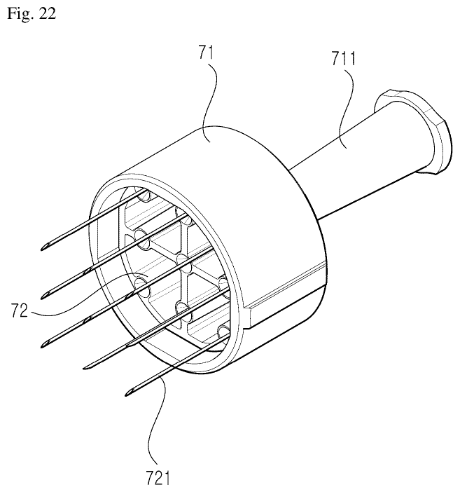

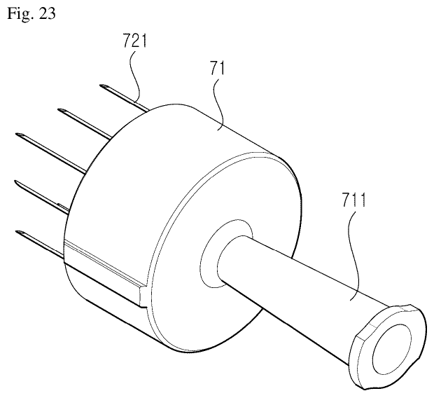

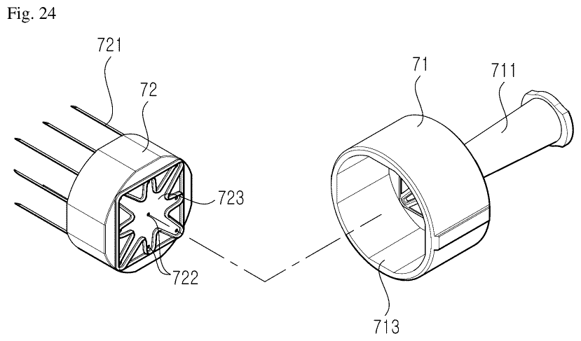

FIG. 18 is a perspective view showing the front of a drug injector according to an embodiment. FIG. 19 is a perspective view showing the rear of a drug injector according to an embodiment. FIG. 20 is an exploded perspective view showing a drug injector with a support frame separated from a needle cover according to an embodiment. FIG. 21 is an exploded perspective view showing a drug injector according to an embodiment. FIG. 22 is a perspective view showing the front of a needle frame according to an embodiment. FIG. 23 is a perspective view showing the rear of a needle frame according to an embodiment. FIG. 24 is a perspective view showing a needle housing before assembly according to an embodiment.

Referring to FIGS. 18 to 24, the structure of the drug injector 40 according to an embodiment will be described.

Referring to FIGS. 18 to 24, according to an embodiment, a drug injector 70 is an injection device that can quantitatively inject drugs into skin cells in environments such as hospitals, homes, and emergency situations, and may be, in particular, a drug injector that is installed in a gun-type device that can inject expensive drugs in quantitative amounts. According to an embodiment, the drug injector 70 may be a drug injector that can inject a fixed amount of drug into the human skin, which is a free surface, using a suction device. For example, the drug injector 70 may be an injector used for intravenous injection, subcutaneous injection, or intramuscular injection.

According to an embodiment, the drug injector 70 may include a cylindrical needle housing 71, a cylindrical needle frame 72, a cylindrical needle cover 73, a support frame 74 and a stopper cap 75. According to an embodiment, the needle frame 72 may have a plurality of needles 721 arranged in an open space on one side, and a drug passage 723 on the other side through which a supplied drug flows. For example, the arrangement of the needles 721 need not be limited to a 9-pin type, and may be changed to a 5-pin type.

According to an embodiment, the drug passage 723 is formed in a grooved cross shape and is connected to each needle hole 722, so that the supplied drug can flow to each needle 721. According to an embodiment, the needle frame 72 is provided with five needle holes 722, and each needle 721 may be fixed to each needle hole 722.

According to an embodiment, the needle housing 71 is a housing member for accommodating the needle frame 72 within an interior 713 thereof, and may have one side open to accommodate the needle frame 72 while the other side formed with an injection part 711 that is physically connected to a syringe. According to an embodiment, the needle housing 71 may be inserted into and restrained by the needle cover 73.

According to an embodiment, the needle cover 73 has a plurality of needle guides 733 arranged in a protruding shape on one side to support the each individual needle 721, and the other side is open so that the needle housing 71 into which the needle frame 72 is inserted and the stopper cap 75 may be accommodated. Each needle guide 733 may have a through hole 734 through which the needle 721 passes.

According to an embodiment, the needle cover 73 may be formed with elastic hook parts 731 facing each other.

According to an embodiment, the needle cover 73 may include a downwardly extending connection part 732. According to an embodiment, the connection part 732 may be physically connected and spatially connected to a duct structure (not shown).

According to an embodiment, when the needle cover 73 comes into contact with the skin in a state where the needle housing 71 with the needle frame 72 combined is inserted into the needle cover 73, a sealed front space 735 is provided between the front surface 730 of the needle cover 73 and the skin, and the sealed front space 735 may converted into a suction space by a suction device.

FIG. 19 is a perspective view showing the rear of a drug injector according to an embodiment. FIG. 20 is an exploded perspective view showing a drug injector with a support frame separated from a needle cover according to an embodiment.

Referring to FIGS. 19 and 20, according to an embodiment, the injection part 711 of the drug injector may be combined with the support frame 74 to safely connect the connection structure for convenience when connecting with a syringe.

According to an embodiment, the support frame 74 is coupled to the needle cover 73 and the injection part 711 to support the connection structure with the syringe. According to an embodiment, the support frame 74 may have a first to third part in the shape of the letter “⊥” when viewed from the front.

According to an embodiment, the support frame 74 may include: a first part 741 that is joined to surround at least a portion of the outer surface of the needle cover 73; a second part 742 formed integrally with the first part 741 and covering at least a portion of the rear surface of the needle cover 73; and a third part 743 formed horizontally as an integral part of the second part 742 and including an opening 744 into which the injection part is inserted and supported.

According to an embodiment, the opening 744 is formed in a portion bent 90 degrees from the third part 743, and a tension part 745 may be provided on each side of the opening 744.

According to an embodiment, the support frame 74 may include a fourth part 746 that is oriented perpendicularly to the third part 743, and the fourth part 746 may be located in the middle of the third part 743.

According to an embodiment, the second part 742 may have a tension protrusion 742a formed on each side thereof. According to an embodiment, the tension protrusions 742a may structurally provide tension when the first part 741 is coupled to the needle cover 73.

FIG. 21 is an exploded perspective view showing a drug injector according to an embodiment.

Referring to FIG. 21, according to an embodiment, the stopper cap 75 is hollow and may be placed between the back of the needle housing 71 and the other end of the needle cover 73. According to an embodiment, the stopper cap 75 is placed at the other end of the needle cover 73, thereby preventing the needle housing 71 from being separated from the needle cover 73. According to an embodiment, the stopper cap 75 may have at least one catch protrusion 751 on the outer surface thereof to be coupled to the needle cover 73. According to an embodiment, the stopper cap 75 may have a through opening 752 formed in the center, through which the injection part 711 may pass. According to an embodiment, the stopper cap 75 may have at least one stopper 753 formed on the outer surface thereof to prevent rotation, and the stopper 753 may protrude in the outer direction.

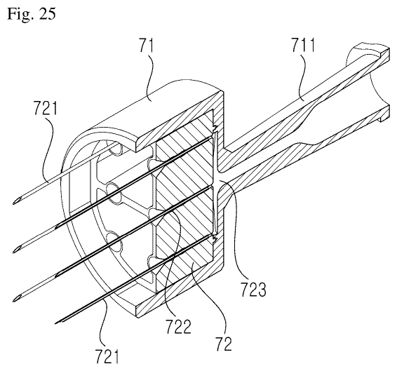

FIG. 25 is a cross-sectional view showing a combined state of a needle frame and a needle housing after assembly according to an embodiment.

Referring to FIG. 25, according to an embodiment, the needle housing 71 may be supplied with a drug through the injection part 711 connected to a syringe. According to an embodiment, the drug supplied through the injection part 711 flows into five needle holes 722 through the drug passage 723 formed on the other surface of the needle frame 72, and the drug flowing into each needle hole 722 may be supplied to each needle 721.

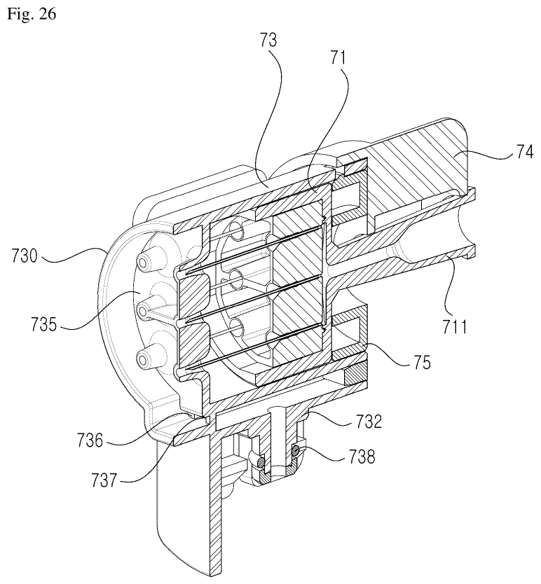

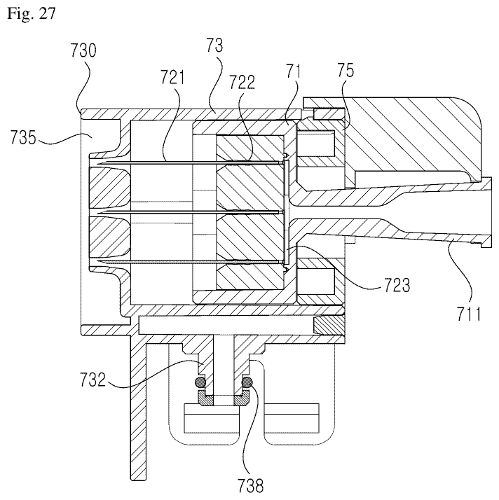

FIGS. 26 and 27 are cross-sectional views each showing an assembled drug injector according to an embodiment.

Referring to FIGS. 26 and 27, when the drug injector 70 comes into contact with the skin, that is, when the front surface 730 of the needle cover 73 comes into contact with the skin, the front space 735 is sealed by the skin. According to an embodiment, the front space 735 is a space where suction is possible, and a path for suction may be formed using a suction device. According to an embodiment, a flow path hole 737 is formed in a wall 736 between the front space 735, which is formed by contact with the skin and the front surface 730 of the needle cover 73, and the connection part 732, so that a path through which suction air flows may be formed. According to an embodiment, the path is a pathway through which suction air flows from the front space 735 to the connection part 732, and may be used as a path for suction. Reference numeral 738 may be a waterproof member.

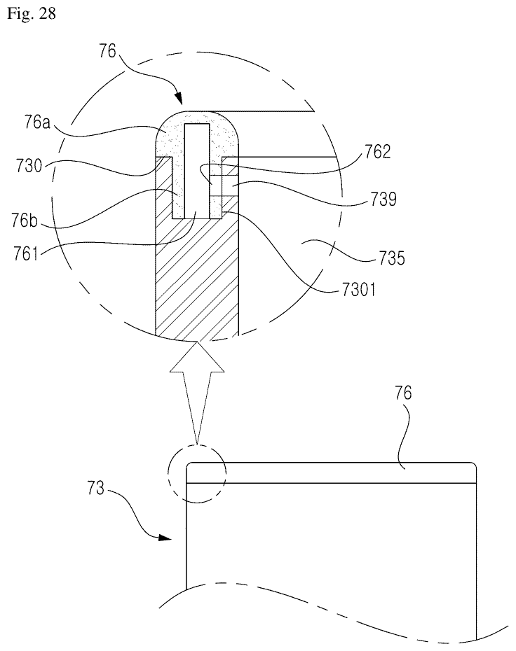

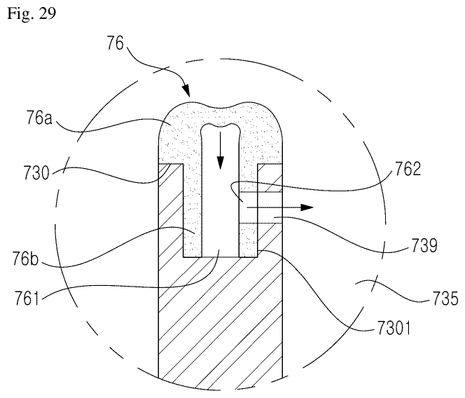

Referring to FIGS. 28 and 29, the drug injector according to an embodiment may have a contact elastic member 76 placed in the front surface 730 of the needle cover 73 to maximize adhesion to the skin and inject expensive, quantitative drugs.

According to an embodiment, an insertion groove 7301 for arranging the contact elastic member 76 may be formed on the front surface 730 of the needle cover 73. According to an embodiment, the insertion groove 7301 may be a ring shape recessed in the front surface 730 of the needle cover. According to an embodiment, the contact elastic member 76 may include an insertion part 76b inserted into the insertion groove 7301; and a contact part 76a that comes into contact with the skin and is integral with the insertion part 76b. According to an embodiment, the contact elastic member 76 is hollow and may include an interior space 761 where suction is possible. According to an embodiment, the insertion part 76b has a shape extending linearly from the contact part 76a, and the contact part 76a has a cross-section that is approximately semi-circular or semi-elliptical so as to have enhanced good adhesion to the skin.

According to an embodiment, when the contact elastic member 76 is inserted into the front surface 730 of the needle cover, a connection structure may be formed for spatially connecting the front space 735 of the needle cover and the interior space 761 of the contact elastic member 76.

According to an embodiment, the connection structure may include: a first connection hole 739 formed in the needle cover; and a second connection hole 762 formed in the insertion part 76b of the contact elastic member and spatially connected to the first connection hole 739. According to an embodiment, when the front space 735 of the needle cover is suctioned, the interior space 761 of the contact elastic member is also structured to be in a state where suction is possible, so that the central portion of the contact part 76a of the contact elastic member may be deformed. According to an embodiment, due to the deformation of the contact part 76a, the contact elastic member may improve the adhesion to the skin, thereby enabling quantitative injection of a drug. For example, the central portion may have a concave shape.

A drug injection device (the drug injection device 10 illustrated in FIG. 1) according to an embodiment may include: a gun-type housing (e.g., the gun-type housing 20 illustrated in FIG. 1) configured to include a grip part (e.g., the grip part 21 illustrated in FIG. 1), an operation part (e.g., the operation part 22 illustrated in FIG. 1) adjacent to the grip part (e.g., the grip part 21 illustrated in FIG. 1), and a mounting part (e.g., the mounting part 23 illustrated in FIG. 1) located above the grip part, and to have an interior space (e.g., the interior space s as shown in FIG. 3) that is spatially connectable with a suction device (e.g., the suction device 60 illustrated in FIG. 16); a syringe (e.g., the syringe 30 illustrated in FIG. 1) placed on the mounting part (e.g., the mounting part 23 illustrated in FIG. 1); a drug injector (e.g., the drug injector 40 illustrated in FIG. 1) that is mounted on an edge of the mounting part (e.g., the mounting part 23 illustrated in FIG. 1), that is capable of supplying a drug by communicating with the syringe (e.g., the syringe 30 illustrated in FIG. 1), that includes a flow path spatially connected to the interior space (e.g., the interior space s illustrated in FIG. 3) of the gun-type housing (e.g., the gun-type housing 20 illustrated in FIG. 1), and that is capable of quantitatively injecting a drug by improving adhesion to the skin due to a vacuum generation part provided between the skin and the front surface by the operation of the suction device (e.g., the suction device 60 illustrated in FIG. 16); a suction connection part (e.g., the suction connection part 26 illustrated in FIG. 1) that is disposed at an edge of the gun-type housing (e.g., the gun-type housing 20 illustrated in FIG. 1) and is spatially connectable with the suction device (e.g., the suction device 60 illustrated in FIG. 16); and an air filtering unit (e.g., the air filtering unit 50 illustrated in FIG. 2) disposed in the gun-type housing (e.g., the gun-type housing 20 illustrated in FIG. 1) and having at least a portion thereof spatially connected to the flow path to filter suction air in the interior space (e.g., the internal space s illustrated in FIG. 3).

According to an embodiment, the air filtering unit (e.g., the air filtering unit 50 illustrated in FIG. 2) may include: a first part (e.g., the first part 51 illustrated in FIG. 3) disposed in the gun-type housing (e.g., the gun-type housing 20 illustrated in FIG. 1); and a second part (e.g., the second part 52 and 53 illustrated in FIG. 3) disposed in the interior space (e.g., the internal space s illustrated in FIG. 3) of the gun-type housing (e.g., the gun-type housing 20 illustrated in FIG. 1) spatially connected to the first part (e.g., the first part 51 illustrated in FIG. 3) and spatially connected to the flow path.

According to an embodiment, the second part (e.g., the second part 52 and 53 illustrated in FIG. 3) is in the shape of a tube, and may include: a first tubular part (e.g., the first tubular part 52 shown in FIG. 3) arranged to face the suction connection part (e.g., the suction connection part 26 illustrated in FIG. 1); and a second tubular part (e.g., the second tubular part 53 illustrated in FIG. 3) arranged to face the drug injector (e.g., the drug injector 40 illustrated in FIG. 1) and spaced apart from the first tubular part.

According to an embodiment, the front space (e.g., the front space 435 illustrated in FIG. 12) of the drug injector (e.g., the drug injector 40 illustrated in FIG. 1) becomes a sealed space when in contact with the skin and becomes a vacuum generation part, and may be converted into a suction space by the operation of the suction device (e.g., the suction device 60 illustrated in FIG. 16).

According to an embodiment, the drug injector (e.g., the drug injector 40 illustrated in FIG. 1) may further include a duct structure (e.g., the duct structure 25 illustrated in FIG. 13B) physically and spatially connected to the flow path.

According to an embodiment, the duct structure (e.g., the duct structure 25 illustrated in FIG. 13B) may be arranged at an edge (e.g., an edge of the mounting part 23 illustrated in FIG. 1 (e.g., the edge 230 of the mounting part illustrated in FIG. 3)), and may be coupled to the connecting part of the drug injector (e.g., a drug injector 40 illustrated in FIG. 1).

According to an embodiment, a waterproof structure may be further arranged between the connecting part and the duct structure (e.g., the duct structure 25 illustrated in FIG. 13B).

According to an embodiment, the waterproof structure may include: a waterproof member placed under pressure on the outer surface of the connection part; and a seating recess formed on the inner surface of the inlet part of the duct structure (e.g., the duct structure 25 illustrated in FIG. 13B) and in which the waterproof member is seated.

According to an embodiment, the duct structure (e.g., the duct structure 25 illustrated in FIG. 13B) may further include a flow path part extending to the air filtering unit (e.g., air filtering unit 50 illustrated in FIG. 2)

According to an embodiment, the duct structure (e.g., the duct structure 25 illustrated in FIG. 13B) may include: a step formed on the outer surface of the flow path part to prevent excessive insertion into the edge of the mounting part (e.g., the mounting part 23 illustrated in FIG. 1); and a catch protrusion formed on the outer surface of the flow path part and spaced apart from the step, and coupled to the edge of the mounting part (e.g., the mounting part 23 illustrated in FIG. 1).

According to an embodiment, the needle cover includes a contact elastic member on the front side thereof to improve adhesion to the skin. The contact elastic member is hollow with an interior space, and may include: a contact part that comes into contact with the skin, in which changes occur in the central portion thereof under suction, and that has a cross-section that is a semicircle or semi-elliptical shape; and an insertion part extending in a straight line from the contact part and inserted into the needle cover.

According to an embodiment, the connection structure may include: a first connection hole formed in the needle cover; and a second connection hole formed in the insertion part and spatially connected to the first connection hole.

A drug injection device (the drug injection device 10 illustrated in FIG. 1) according to an embodiment may include: a gun-type housing (e.g., the gun-type housing 20 illustrated in FIG. 1) configured to include a grip part (e.g., the grip part 21 illustrated in FIG. 1), an operation part (e.g., the operation part 22 illustrated in FIG. 1) adjacent to the grip part (e.g., the grip part 21 illustrated in FIG. 1), and a mounting part (e.g., the mounting part 23 illustrated in FIG. 1) located above the grip part, and to have an interior space (e.g., the interior space s as shown in FIG. 3) that is spatially connected with a suction device (e.g., the suction device 60 illustrated in FIG. 16); a syringe (e.g., the syringe 30 illustrated in FIG. 1) placed on the mounting part (e.g., the mounting part 23 illustrated in FIG. 1); a drug injector (e.g., the drug injector 40 illustrated in FIG. 1) that is mounted on an edge of the mounting part (e.g., the mounting part 23 illustrated in FIG. 1), that is capable of supplying a drug by communicating with the syringe (e.g., the syringe 30 illustrated in FIG. 1), that includes a flow path spatially connected to the interior space (e.g., the interior space s illustrated in FIG. 3) of the gun-type housing (e.g., the gun-type housing 20 illustrated in FIG. 1), and that is capable of quantitatively injecting a drug by improving adhesion to the skin due to a vacuum generation part provided between the skin and the front surface by the operation of the suction device (e.g., the suction device 60 illustrated in FIG. 16); a suction connection part (e.g., the suction connection part 26 illustrated in FIG. 1) that is disposed at an edge of the gun-type housing (e.g., the gun-type housing 20 illustrated in FIG. 1) and is connectable with the suction device (e.g., the suction device 60 illustrated in FIG. 16); an air filtering unit (e.g., the air filtering unit 50 illustrated in FIG. 2) disposed in the gun-type housing (e.g., the gun-type housing 20 illustrated in FIG. 1), having at least one end thereof positioned in the interior space (e.g., the interior space s illustrated in FIG. 3), spatially connected to the flow path, and configured to filter suction air in the interior space (e.g., the internal space s illustrated in FIG. 3); and at least one air tube physically and spatially connecting between the suction connection part, the air filtering unit (e.g., the air filtering unit 50 illustrated in FIG. 2) and the drug injector (e.g., the drug injector 40 illustrated in FIG. 1).

According to an embodiment, the air tube may further include a first air tube that physically and spatially connects the suction connection part and the air filtering unit (e.g., the air filtering unit 50 illustrated in FIG. 2).

According to an embodiment, the air tube may further include a second air tube that physically and spatially connects the air filtering unit (e.g., the air filtering unit 50 illustrated in FIG. 2) and the drug injector (e.g., the drug injector 40 illustrated in FIG. 1)

According to an embodiment, the drug injector (e.g., the drug injector 40 illustrated in FIG. 1) may further include a duct structure (e.g., the duct structure 25 illustrated in FIG. 13B) spatially connected to the front space (e.g., the front space 435 illustrated in FIG. 12), and the other end of the second air tube may be physically and spatially connected to the duct structure (e.g., the duct structure 25 illustrated in FIG. 13B).

According to an embodiment, the drug injector may include: a cylindrical needle frame including a plurality of needles; a cylindrical needle housing that accommodates the needle frame and includes an injection part connected to a syringe; a cylindrical needle cover into which the needle housing is inserted, having a plurality of needle guides supporting the plurality of needles, that improves adhesion to the skin by forming a vacuum generation part between the skin and the front surface thereof, thereby enabling quantitative drug injection, and having at least one hook part formed; a support frame assembled in a portion of the needle cover to support the connection state of the injection part and the syringe; and a stopper cap that is attached to the needle cover and prevents the needle housing from being separated from the needle cover.

According to an embodiment, the support frame may include: a first part that is joined to surround at least a portion of the outer surface of the needle cover; a second part formed integrally with the first part and covering at least a portion of the rear surface of the needle cover; and a third part formed horizontally as an integral part of the second part and including an opening into which the injection part is inserted and supported.

According to an embodiment, the opening is formed in a portion bent 90 degrees from the third part, and a tension part may be provided on each side of the opening.

According to an embodiment, the support frame may include a fourth part that is oriented perpendicularly to the third part, and the fourth part may be located in the middle of the third part.

According to an embodiment, the second part may have a tension protrusion formed on each side thereof.

According to an embodiment, the stopper cap is hollow and may be placed between the back of the needle frame and the other end of the needle cover.

According to an embodiment, the stopper cap may have at least one catch protrusion formed on the outer surface thereof to be joined to the needle cover, and has a through opening formed in the center thereof, through which the injection part can pass.

According to an embodiment, the stopper cap may have at least one stopper formed on the outer surface thereof to prevent rotation, and the stopper may protrude in the direction of the outer periphery of the stopper cap.

In this disclosure, “comprising at least one of a, b, or c” may mean “comprising only a, comprising only b, comprising only c, or comprising a combination of two or more of them (comprising a and b, comprising b and c, comprising a and c, or comprising all of a, b, and c)”.

Meanwhile, although specific embodiments have been described above in the detailed description of the present disclosure, it should be understood that various modifications may be made without departing from the scope of the present disclosure. Therefore, the scope of the present disclosure should not be limited to the described embodiments, but should be determined not only by the scope of the claims described below, but also by equivalents of the scope of these claims.

What is claimed is:

1. A drug injection device comprising:
a gun-type housing configured to include a grip part, an operation part adjacent to the grip part, and a mounting part located above the grip part, and to have an interior space that is spatially connectable with a suction device;
a syringe placed on the mounting part;
a drug injector mounted on an edge of the mounting part, configured to supply a drug by communicating with the syringe, provided with a flow path spatially connected to the interior space of the gun-type housing, and configured to quantitatively inject a drug by improving adhesion to skin due to a vacuum generation part provided between the skin and a front surface thereof by an operation of the suction device;
a suction connection part disposed at an edge of the gun-type housing and spatially connectable with the suction device; and
an air filtering unit disposed in the gun-type housing and configured to have at least a portion thereof spatially connected to the flow path to filter suction air in the interior space.

2. The device of claim 1, wherein the air filtering unit comprises:
a first part arranged in the gun-type housing; and
a second part arranged in the interior space of the gun-type housing, spatially connected to the first part, and spatially connected to the flow path.

3. The device of claim 2, wherein the second part has a tube shape, and comprises: a first tubular part arranged to face the suction connection part; and a second tubular part arranged to face the drug injector and spaced apart from the first tubular part.

4. The device of claim 1, wherein a front space of the drug injector becomes a sealed space when in contact with the skin, and can be converted into a suction space by the operation of the suction device.

5. The device of claim 1, wherein the drug injector further comprises a duct structure physically and spatially connected to the flow path.

6. The device of claim 5, wherein the duct structure is placed at the edge of the mounting part and is connected to a connection part of the drug injector.

7. The device of claim 6, wherein a waterproof structure is further arranged between the connection part and the duct structure.

8. The device of claim 7, wherein the waterproof structure comprises:
a waterproof member placed under pressure on an outer surface of the connection part; and
a seating recess provided on an inner surface of an inlet part of the duct structure and in which the waterproof member is seated.

9. The device of claim 5, wherein the duct structure further comprises a flow path part extending to the air filtering unit.

10. The device of claim 5, wherein the duct structure comprises:
a step provided on an outer surface of a flow path part to prevent excessive insertion into the edge of the mounting part; and
a catch protrusion provided on the outer surface of the flow path part, spaced apart from the step, and coupled to the edge of the mounting part.

11. The device of claim 1, wherein the drug injector comprises:
a cylindrical needle frame configured to include a plurality of needles;
a cylindrical needle housing configured to accommodate the needle frame and to include an injection part connected to a syringe;
a cylindrical needle cover into which the needle housing is inserted, configured to have a plurality of needle guides supporting the plurality of needles, configured to improve adhesion to skin by forming a vacuum generation part between the skin and a front surface thereof, thereby enabling quantitative drug injection, and configured to have at least one hook part;
a support frame assembled in a portion of the needle cover to support a connection state of the injection part and the syringe; and
a stopper cap attached to the needle cover and configured to prevent the needle housing from being separated from the needle cover.

12. The device of claim 11, wherein the support frame comprises:
a first part that is joined to surround at least a portion of an outer surface of the needle cover;
a second part formed integrally with the first part and covering at least a portion of a rear surface of the needle cover; and
a third part formed horizontally as an integral part of the second part and including an opening into which the injection part is inserted and supported.

13. The device of claim 12, wherein the opening is formed in a portion bent 90 degrees from the third part, and is provided with a tension part on each side thereof.

14. The device of claim 12, wherein the support frame comprises a fourth part that is oriented perpendicularly to the third part,
wherein the fourth part is located in a middle of the third part.

15. The device of claim 12, wherein the second part is provided with a tension protrusion on each side thereof.

16. The device of claim 11, wherein the stopper cap is hollow and is placed between a back of the needle frame and a second end of the needle cover.

17. The device of claim 11, wherein the stopper cap has at least one catch protrusion formed on an outer surface thereof so as to be joined to the needle cover, and is provided with a through opening in a center thereof, through which the injection part passes.

18. The device of claim 17, wherein the stopper cap has at least one stopper formed on the outer surface thereof to prevent rotation, and the stopper protrudes in a direction of an outer periphery of the stopper cap.

19. The device of claim 11, wherein the needle cover comprises a contact elastic member on a front side thereof to improve adhesion to the skin,
wherein the contact elastic member is hollow and has an interior space, and comprises:
a contact part that comes into contact with the skin, in which changes occur in a central portion thereof under suction, and that has a cross-section that is a semicircle or semi-elliptical shape; and
an insertion part extending in a straight line from the contact part and inserted into the needle cover.

20. The device of claim 19, wherein a connection structure is formed so that a front space of the needle cover and the interior space of the contact elastic member are spatially connected,
wherein the connection structure comprises:
a first connection hole formed in the needle cover; and
a second connection hole formed in the insertion part and spatially connected to the first connection hole.
